(19) United States
(12) Patent Application Publication
(10) Pub. No.: US 2004/0241242 A1
(43) Pub. Date: Dec. 2, 2004

(54) Thin film delivery systems for volatile decongestants

(75) Inventors: Richard C. Fuisz, McLean, VA (US); Robert K. Yang, Flushing, NY (US); Gary L. Myers, Kingsport, TN (US)

Correspondence Address: HOFFMANN & BARON, LLP, 6900 JERICHO TURNPIKE, SYOSSET, NY, 11791, US

(73) Assignee: KOSMOS PHARMA

(21) Appl. No.: 10/730,347

(22) Filed: Dec. 5, 2003

Related U.S. Application Data
(60) Provisional application No. 60/431,589, filed on Dec. 6, 2002.

(51) Int. Cl. A61K 9/14

(52) U.S. Cl. 424/486; 424/488

(57) ABSTRACT

A volatile decongestant delivery vehicle composition includes (i) a flowable water-soluble film-forming matrix; and (ii) a particulate volatile decongestant agent uniformly stationed therein. A useful volatile decongestant agent includes menthol, for example menthol crystals. The composition may further include a decongesting volatile oil, such as, but not limited to eucalyptus oil, menthol oil, pine oil, terpine hydrate oil, and combinations thereof. The volatile decongestant agent may be present in amounts of up to about 0.1% to about 60% by weight of the total composition.

### FIELD OF THE INVENTION

The present invention relates to compositions and methods for the preparation and use of a uniform rapid dissolve dosage form in the form of a film that includes a volatile decongestant.

### BACKGROUND OF RELATED TECHNOLOGY

Aromatic vapors from topically applied camphor, menthol and eucalyptus oils tend to reduce nasal airflow resistance and reduce congestion. The vapors may also be used as a cough suppressant.

Lozenges containing menthol are readily available for nonprescription use. Decongestant formulations containing menthol for direct application to nasal passages have been proposed. For example, U.S. Pat. No. 4,927,631 discloses a decongestant preparation of a petrolatum base and a mixture of active components consisting of menthol, camphor, eucalyptus oil and spirits of turpentine. The amount of active components is limited to less than one percent, which limits the preparation's decongestant capabilities.

There have been several attempts to provide an alternate dosage form, such as a film that would include a pharmaceutical active. However, such attempts have not been successful in providing a film that incorporates a drug with sufficient uniformity to provide accurate dosing.

Films that incorporate a pharmaceutically active ingredient are disclosed in expired U.S. Pat. No. 4,136,145 to Fuchs, et al. (“Fuchs”). These films may be formed into a sheet, dried and then cut into individual doses. The Fuchs disclosure alleges the fabrication of a uniform film, which includes the combination of water-soluble polymers, surfactants, flavors, sweeteners, plasticizers and drugs. These allegedly flexible films are disclosed as being useful for oral, topical or enteral use. Examples of specific uses disclosed by Fuchs include application of the films to mucosal membrane areas of the body, including the mouth, rectal, vaginal, nasal and ear areas.

Examination of films made in accordance with the process disclosed in Fuchs, however, reveals that such films suffer from the aggregation or conglomeration of particles, i.e., self-aggregation, making them inherently non-uniform. This result can be attributed to Fuchs' process parameters, which although not specifically disclosed likely include the use of relatively long drying times, thereby facilitating intermolecular attractive forces, convection forces, air flow and the like to form such agglomeration. Moreover, Fuchs fails to describe films having volatile components, including films having volatile components as predominant active ingredients.

Thymol, methyl salicylate, eucalyptol and menthol oils have been added to orally consumable films. For example, WO 00/18365 describes films containing pullulan and combinations of these oils to provide a film useful as a breath freshener. The use of such oils, however, limits the overall quantity of the oils that can be placed in a film to about 15 weight percent, as limited by film processing or film integrity concerns. This reference, however, fails to disclose consumable films containing a sufficient quantity of menthol suitable for decongestant purposes.

Therefore, there is a need for a rapid dissolve dosage form, presented as a uniform film that addresses and corrects the problems associated with non-uniformity of active components in the film. Moreover, there is a need for a film having volatile components in sufficient quantities for use as a decongestant.

### SUMMARY OF THE INVENTION

The present invention seeks to attain low adjuvant content, volatile decongestant-containing films which have enhanced flexibility, structural integrity and uniformity. The present invention also provides for a unique method of producing the inventive compositions such that the compositional components are evenly distributed throughout the film. This process is described in detail in co-pending U.S. patent application Ser. No. 10/074,272 and PCT Patent Application No. PCT/US02/32,575, entitled “Thin Film with Non-Self-Aggregating Uniform Heterogeneity and Drug Delivery Systems Made Therefrom”, the subject matter of which is herein incorporated by its entirety.

In one aspect of the present invention, a volatile decongestant delivery vehicle composition includes, but is not limited to, a flowable water-soluble film-forming matrix; and a particulate volatile decongestant agent uniformly stationed therein.

The matrix may be a cellulosic material, a gum, a protein, a starch, a glucan, and combinations thereof. For example, useful material for the matrix include, but not limited to, cellulosic materials, such as carboxymethyl cellulose, methyl cellulose, ethyl cellulose, hydroxyl methyl cellulose, hydroxyethyl cellulose, hydroxypropyl cellulose, hydroxypropylmethyl cellulose, hydroxymethylpropyl cellulose, and combinations thereof; gums, such as gum arabic, xanthan gum, tragacanth, acacia, carageenan, guar gum, locust bean gum, pectin, alginates and combinations thereof; starches, such as tapioca, rice, corn, potato, wheat and combinations thereof; polyvinyl alcohol; polyacrylic acid; polyvinyl pyrrolidone; poly(meth)acrylate; poly(meth)copolymers; and proteins, such as gelatin, zein, gluten, soy protein, soy protein isolate, whey protein, whey protein isolate, casein, levin, collagen and combinations thereof; dextrin; dextran; chitin; chitosin; polydextrose; fructose oligomers; and combinations thereof.

In one aspect of the present invention, the volatile decongestant agent is menthol. Desirably, the volatile decongestant agent is menthol crystals. Additional decongesting agents may suitably be used, volatile oils, such as eucalyptus oil, menthol oil, pine oil, or terpine hydrate oil. The volatile decongestant agent, including volatile oils, may be present in amounts of up to about 0.1% to about 60% by weight of the total composition.

The delivery vehicle composition of the present invention may be orally or nasally deliverable. The delivery composition of the present invention may be essentially free of a surfactant, essentially free of a plasticizer or essentially free of a polyalcohol.

In another aspect of the present invention, a method of preparing a thin film volatile decongestant delivery vehicle is provided. The method includes the steps of providing a volatile decongestant agent complex; combining the complex with a water-soluble polymer and a solvent to form a decongestant mixture with uniform distribution of the complex therein; casting the mixture onto a planar carrier surface to form a thin film on the carrier surface; and controllably drying the thin film to form a distribution variance of the complex having less than about 10% variance throughout any given area of the thin film. The method of the present invention may include applying heat to the bottom of the carrier surface or applying microwave energy to the film to dry the film. Desirably, the mixing of the water-soluble polymer and the solvent is performed to form a pre-decongestant mixture of uniform distribution. The volatile decongestant may be added after mixing the pre-decongestant mixture with the time of mixing the pre-decongestant mixture being greater than the time of mixing the decongestant mixture thereinto. The decongestant mixture desirably includes menthol crystals and may further include a decongesting volatile oil.

Articles of manufacture containing the inventive decongestant films of the present invention and methods of use are also provided.

### BRIEF DESCRIPTION OF THE DRAWINGS

FIG. 1 shows a side view of a package containing a unit dosage film of the present invention.

FIG. 2 shows a top view of two adjacently coupled packages containing individual unit dosage forms of the present invention, separated by a tearable perforation.

FIG. 3 shows a side view of the adjacently coupled packages of FIG. 2 arranged in a stacked configuration.

FIG. 4 shows a perspective view of a dispenser for dispensing the packaged unit dosage forms, dispenser containing the packaged unit dosage forms in a stacked configuration.

FIG. 5 is a schematic view of a roll of coupled unit dose packages of the present invention.

FIG. 6 is a schematic view of an apparatus suitable for preparation of a pre-mix, addition of an active, and subsequent formation of the film.

FIG. 7 is a schematic view of an apparatus suitable for drying the films of the present invention.

FIG. 8 is a cross-sectional view of a volatile decongestant-containing film of the present invention contained within a package.

FIG. 9 is a cross-sectional view of the film of FIG. 8 contained between films not containing volatile decongestants.

FIG. 10 is a cross-sectional view of the film of FIG. 9 encased within films not containing volatile decongestants.

### DETAILED DESCRIPTION OF THE INVENTION

The present invention provides a decongestant composition in the form of a film for external or topical administration, including a composition having a uniformly distributed combination of a polymer, a polar solvent, and a taste-masked pharmaceutically active or bioeffecting agent. The composition in its dried film form maintains the uniform distribution of components through the application of controlled bottom drying of the film.

Aromatic vapors from topically applied menthol crystals, menthol oil, camphor and eucalyptus oil have antitussive, anesthetic, analgesic, antipruritic and decongestant activity. In particular, menthol vapors reduce nasal airflow resistance and congestion. Menthol vapors also act as a cough suppressant and act to relieve a sore throat.

Menthol crystals are produced by extraction of menthol from menthol oil via fractionation and separation by crystallization. Menthol crystals, i.e., 1-methyl-4-isopropyl cyclohexane-3-ol, are typically 97 percent or greater in purity. Menthol crystals have a melting point of about 41-44° C., depending upon purity. Menthol oils, such as those oils derived from peppermint oils, often containing only from 30 to 80% menthol.

The decongestant films of the present invention contain menthol crystals uniformly dispersed in water-soluble polymers. The decongestant films may also contain other volatile oils, such as eucalyptus oil, menthol oil, pine oil and terpine hydrate oil. The use of menthol crystals, however, provide advantages over prior art attempts to incorporate volatile oils into films. The use of menthol crystals permits greater menthol loading and improved film properties when the films are prepared in accordance with the methods of the present invention.

Water-soluble polymers useful in the present invention include cellulosic materials, gums, proteins, starches, and combinations thereof.

As used herein the phrase “water soluble polymer” and variants thereof refer to a polymer that is at least partially soluble in water, and desirably fully or predominantly soluble in water, or absorbs water. Polymers that absorb water are often referred to as being water swellable polymers. The materials useful with the present invention may be water soluble or water swellable at room temperature and other temperatures, such as temperatures exceeding room temperature. Moreover, the materials may be water soluble or water swellable at pressures less than atmospheric pressure. Desirably, the water soluble polymers are water soluble or water swellable having at least 20 percent by weight water uptake. Water swellable polymers having a 25 or greater percent by weight water uptake are also useful. Films or dosage forms of the present invention formed from such water soluble polymers are desirably sufficiently water soluble to be dissolvable upon contact with bodily fluids.

Examples of cellulosic materials include, without limitation, carboxymethyl cellulose, methyl cellulose, ethyl cellulose, hydroxylmethyl cellulose, hydroxyethyl cellulose, hydroxypropyl cellulose, hydroxypropylmethyl cellulose, hydroxymethylpropyl cellulose, and combinations thereof.

Examples of water-soluble gums include gum arabic, xanthan gum, tragacanth, acacia, carageenan, guar gum, locust bean gum, pectin, alginates and combinations thereof.

Examples of other polymeric materials which may be incorporated include polyvinyl alcohol, polyacrylic acid, polyvinyl pyrrolidone, poly(meth)acrylate, poly(meth)copolymers and combinations thereof.

Useful starches include gelatinized, modified or unmodified starches. The source of the starches may vary and include pullulan, tapioca, rice, corn, potato, wheat and combinations thereof.

Useful water-soluble protein polymers include gelatin, zein, gluten, soy protein, soy protein isolate, whey protein, whey protein isolate, casein, levin, collagen and combinations thereof. Additional water-soluble polymers include dextrin, dextran and combinations thereof, as well as chitin, chitosin and combinations thereof, polydextrose and fructose oligomers.

Although a variety of different polymers may be used, it is desired to select polymers to provide a desired viscosity of the mixture prior to drying. The polymer plays an important role in affecting the viscosity of the film. Viscosity is one property of a liquid that controls the stability of the active in an emulsion, a colloid or a suspension. Generally the viscosity of the matrix will vary from about 400 cps to about 100,000 cps, preferably from about 800 cps to about 60,000 cps, and most preferably from about 1,000 cps to about 40,000 cps. Desirably, the viscosity of the film-forming matrix will rapidly increase upon initiation of the drying process.

The edible water-soluble delivery system of the present invention further includes glucans, such as pullulan and elsinan. The ratio of glucan to water soluble polymer is about 40:1 to about 0.1:5. Glucans are generally desirable materials for edible film because of their high water solubility, rapid dissolution and excellent mouth-feel.

The edible water-soluble delivery system of the present invention further include an anti-foaming or defoaming agent, such as simethicone, which is a combination of a polymethylsiloxane and silicon dioxide. Simethicone acts as either an anti-foaming or defoaming agent which reduces or eliminates air from the film composition. An anti-foaming agent will aid in preventing the introduction of air into a composition, while a defoaming agent will aid in removing air from the composition.

The edible water-soluble delivery system of the present invention further include an active component selected from cosmetic agents, pharmaceutical agents, bioactive agents and combinations thereof. The active component may be present in any amount effective for the intended treatment. It is particularly desirable and an advantage of the present invention that the active component can be included in high loads. For example, the active component may be present in amounts up to about 60% by weight of the total composition and desirably in amounts of 0.01% to about 50% by weight of total composition.

Additionally, organoleptic agents, such as, but not limited to sweeteners and/or flavors, may also be employed in the compositions of the present invention. Suitable sweeteners include both natural and artificial sweeteners. Non-limiting examples of suitable sweeteners include, e.g.:

a. water-soluble sweetening agents such as monosaccharides, disaccharides and polysaccharides such as xylose, ribose, glucose (dextrose), mannose, galactose, fructose (levulose), sucrose (sugar), maltose, invert sugar (a mixture of fructose and glucose derived from sucrose), partially hydrolyzed starch, corn syrup solids, dihydrochalcones, monellin, steviosides, and glycyrrhizin;

b. water-soluble artificial sweeteners such as the soluble saccharin salts, i.e., sodium or calcium saccharin salts, cyclamate salts, the sodium, ammonium or calcium salt of 3,4-dihydro-6-methyl-1,2,3-o xathiazine-4-one-2, 2-dioxide, the potassium salt of 3,4-dihydro-6-methyl-1,2,3-oxathia zine-4-one-2,2-dioxide (acesulfame-K), the free acid form of saccharin and the like;

c. dipeptide based sweeteners, such as L-aspartic acid derived sweeteners, such as L-aspartyl-L-phenylalanine methyl ester (aspartame), L-alpha-aspartyl-N-(2,2,4,4-tetramethyl-3-thietanyl)-D-alaninamide hydrate, methyl esters of L-aspartyl-L-phenylglycerin and L-aspartyl-L-2,5,dihydrophenylglycine, L-aspartyl-2,5-dihydro-L-phenylalanine, L-aspartyl-L-(1-cyclohexyen)-alanine, and the like;

d. water-soluble sweeteners derived from naturally occurring water-soluble sweeteners, such as a chlorinated derivatives of ordinary sugar (sucrose), known, for example, under the product description of sucralose; and

e. protein based sweeteners such as thaurnatoccous danielli (Thaumatin I and II).

In general, an effective amount of auxiliary sweetener is utilized to provide the level of sweetness desired for a particular composition, and this amount will vary with the sweetener selected. This amount will normally be 0.01% to about 10% by weight of the composition. These amounts may be used to achieve a desired level of sweetness independent from the flavor level achieved from any optional flavor oils used. Of course, sweeteners need not be added to films intended for non-oral administration.

Useful flavors or flavoring agents include natural and artificial flavors. These flavorings may be chosen from synthetic flavor oils and flavoring aromatics, and/or oils, oleo resins and extracts derived from plants, leaves, flowers, fruits and so forth, and combinations thereof. Non-limiting flavor oils include: spearmint oil, cinnamon oil, peppermint oil, clove oil, bay oil, thyme oil, cedar leaf oil, oil of nutmeg, oil of sage, and oil of bitter almonds. Also useful are artificial, natural or synthetic fruit flavors such as vanilla, chocolate, coffee, cocoa and citrus oil, including lemon, orange, grape, lime and grapefruit, and fruit essences including apple, pear, peach, strawberry, raspberry, cherry, plum, pineapple, apricot and the like. These flavorings can be used individually or in combination. Commonly used flavors include mints such as peppermint, artificial vanilla, cinnamon derivatives, and various fruit flavors, whether employed individually or in combination. Flavorings such as aldehydes and esters including cinnamylacetate, cinnamaldehyde, citral, diethylacetal, dihydrocarvyl acetate, eugenyl formate, p-methylanisole, and the like may also be used. Further examples of aldehyde flavorings include, but are not limited to acetaldehyde (apple); benzaldehyde (cherry, almond); cinnamicaldehyde (cinnamon); citral, i.e., alpha citral (lemon, lime); neral, i.e. beta citral(lemon, lime); decanal (orange, lemon); ethyl vanillin (vanilla, cream);heliotropine, i.e., piperonal (vanilla, cream); vanillin (vanilla, cream); alpha-amyl cinnamaldehyde (spicy fruity flavors); butyraldehyde (butter, cheese);valeraldehyde (butter, cheese); citronellal (modifies, many types); decanal(citrus fruits); aldehyde C-8 (citrus fruits); aldehyde C-9 (citrus fruits);aldehyde C-12 (citrus fruits); 2-ethyl butyraldehyde (berry fruits); hexenal, i.e. trans-2 (berry fruits); tolyl aldehyde (cherry, almond); veratraldehyde (vanilla); 12,6-dimethyl-5-heptenal, i.e. melonal (melon); 2 dimethyloctanal (greenfruit); and 2-dodecenal (citrus, mandarin); cherry; grape; mixtures thereof; and the like.

The amount of flavoring employed is normally a matter of preference, subject to such factors as flavor type, individual flavor, and strength desired. The amount may be varied in order to obtain the result desired in the final product. Such variations are within the capabilities of those skilled in the art without the need for undue experimentation. In general, amounts of about 0.1 to about 30 wt % are useful with the practice of the present invention.

The edible water-soluble delivery system of the present invention further includes one or more members selected from antifoaming agents, plasticizing agents, surfactants, emulsifying agents, thickening agents, binding agents, cooling agents, saliva-stimulating agents, sweetening agents, antimicrobial agents, antigens and combinations thereof.

In one aspect of the present invention, a volatile decongestant delivery vehicle composition includes (i) a flowable water-soluble film-forming matrix; and (ii) a particulate volatile decongestant agent uniformly stationed therein. Desirably, the volatile decongestant agent is menthol, including menthol crystals. The composition may further include a decongesting volatile oil, such as, but not limited to eucalyptus oil, menthol oil, pine oil, terpine hydrate oil, and combinations thereof. Desirably, the volatile decongestant agent is present in amounts of up to about 0.1% to about 60% by weight of the total composition.

The composition is orally or intranasally deliverable. As such, the volatile decongestant delivery vehicle composition is a dry mucoadhering film. The particulate decongestant agent desirably has a particle size of 200 microns or less, and the flowable water-soluble film-forming matrix is capable of being dried without loss of uniformity in the stationing of the particulate decongestant agent therein.

The matrix may be a cellulosic material, a gum, a protein, a starch, a glucan, and combinations thereof. Desirably, the cellulosic material is carboxymethyl cellulose, methyl cellulose, ethyl cellulose, hydroxyl methyl cellulose, hydroxyethyl cellulose, hydroxypropyl cellulose, hydroxypropylmethyl cellulose, hydroxymethylpropyl cellulose, and combinations thereof. Desirably, the gum is gum arabic, xanthan gum, tragacanth, acacia, carageenan, guar gum, locust bean gum, pectin, alginates and combinations thereof. Desirably, the starch is tapioca, rice, corn, potato, wheat and combinations thereof. The starch may be gelatinized, modified or unmodified. Moreover, useful matrix materials include polyvinyl alcohol, polyacrylic acid, polyvinyl pyrrolidone, poly(meth)acrylate, poly(meth)copolymers and combinations thereof. Useful proteins include gelatin, zein, gluten, soy protein, soy protein isolate, whey protein, whey protein isolate, casein, levin, collagen and combinations thereof. Furthermore, dextrin, dextran and combinations thereof are also useful as matrix materials. Additionally useful matrix materials include chitin, chitosin and combinations thereof and polydextrose, fructose oligomers, and combinations thereof.

The delivery composition may be essentially free of a surfactant, essentially free of a plasticizer, and/or essentially free of a polyalcohol.

In another aspect of the present invention, a method of preparing a volatile decongestant delivery vehicle composition is provided. The method includes the steps of (a) providing a volatile decongestant agent complex; (b) combining the complex with a water-soluble polymer and a solvent to form a decongestant mixture with uniform distribution of the complex therein; (c) casting the mixture onto a planar carrier surface to form a thin film on the carrier surface; and (d) controllably drying the thin film to form a distribution variance of the complex having less than about 10% variance throughout any given area of the thin film.

The method of the present invention includes applying heat to the bottom of the carrier surface, applying microwave energy to the film, and combinations thereof.

The drying includes applying heat to the bottom of the carrier surface. Moreover, the drying may include applying microwave energy to the film. Such microwave drying is useful because drying initiates in the middle portions of the film. The present invention, however, is not limited to these drying methods. Any drying method may suitably be used as long as the drying does not initiate at the top surface of the casted mixture. Such top surface drying does not typically provide desirable film uniformity. Moreover, drying temperatures may be controlled to control the film temperature. For example, the film drying temperature may be controlled to minimize the lose of volatile decongestant components. The film drying temperature may be so controlled by varying the temperature and/or the drying time. The film drying temperature may be at or below the melting temperature of the particulate and volatile decongestants. Alternatively, the film drying temperature may be greater than the melting of the volatile decongestants, but drying residence time in such a case should be reduced to reduce decongestant loss.

The method of the present invention may further include mixing the water-soluble polymer and the solvent to form a pre-decongestant mixture and mixing the pre-decongestant mixture to obtain uniform distribution. Desirably, the decongestant complex is added after mixing said pre-decongestant mixture. Additionally, it is desirable to have the time of mixing the pre-decongestant mixture is greater than the time for mixing the decongestant mixture therein. In other words, quick mixing of the decongestant complex minimized losses of the volatile components.

In another aspect of the present invention, a method of providing decongesting relief is provided. The method includes orally or intranasally delivery of the delivery volatile decongestant vehicle composition of the present invention.

In another aspect of the present invention, a decongestant article is provided. The article includes the delivery volatile decongestant vehicle composition of the present invention and an enclosure for the composition. The enclosure may include a foil, such as a metal foil, encompassing the composition. The enclosure may also include an outer film obtained from a flowable water-soluble film-forming matrix. Desirably, the outer film is essentially free of volatile decongestants.

Uses of Thin Films

The thin films of the present invention are well suited for many uses. The high degree of uniformity of the components of the film makes them particularly well suited for incorporating pharmaceuticals. Desirably, the volatile decongestant film of the present invention is orally or intranasally administered to provide decongesting relief to a person in need of such treatment. Furthermore, the polymers used in construction of the films may be chosen to allow for a range of disintegration times for the films. A variation or extension in the time over which a film will disintegrate may achieve control over the rate that the active is released, which may allow for a sustained release delivery system. In addition, the films may be used for the administration of an active to any of several body surfaces, especially those including mucous membranes, such as oral, anal, vaginal, ophthalmological, the surface of a wound, either on a skin surface or within a body such as during surgery, and similar surfaces.

The films may be used to orally administer an active. This is accomplished by preparing the films as described above and introducing them to the oral cavity of a mammal. This film may be prepared and adhered to a second or support layer from which it is removed prior to use, i.e. introduction to the oral cavity. An adhesive may be used to attach the film to the support or backing material which may be any of those known in the art, and is preferably not water soluble. If an adhesive is used, it will desirably be a food grade adhesive that is ingestible and does not alter the properties of the active. Mucoadhesive compositions are particularly useful. The film compositions in many cases serve as mucoadhesives themselves.

The films may be applied under or to the tongue of the mammal. When this is desired, a specific film shape, corresponding to the shape of the tongue may be preferred. Therefore the film may be cut to a shape where the side of the film corresponding to the back of the tongue will be longer than the side corresponding to the front of the tongue. Specifically, the desired shape may be that of a triangle or trapezoid. Desirably, the film will adhere to the oral cavity preventing it from being ejected from the oral cavity and permitting more of the active to be introduced to the oral cavity as the film dissolves.

Another use for the films of the present invention takes advantage of the films' tendency to dissolve quickly when introduce to a liquid. An active may be introduced to a liquid by preparing a film in accordance with the present invention, introducing it to a liquid, and allowing it to dissolve. This may be used either to prepare a liquid dosage form of an active, or to flavor a beverage.

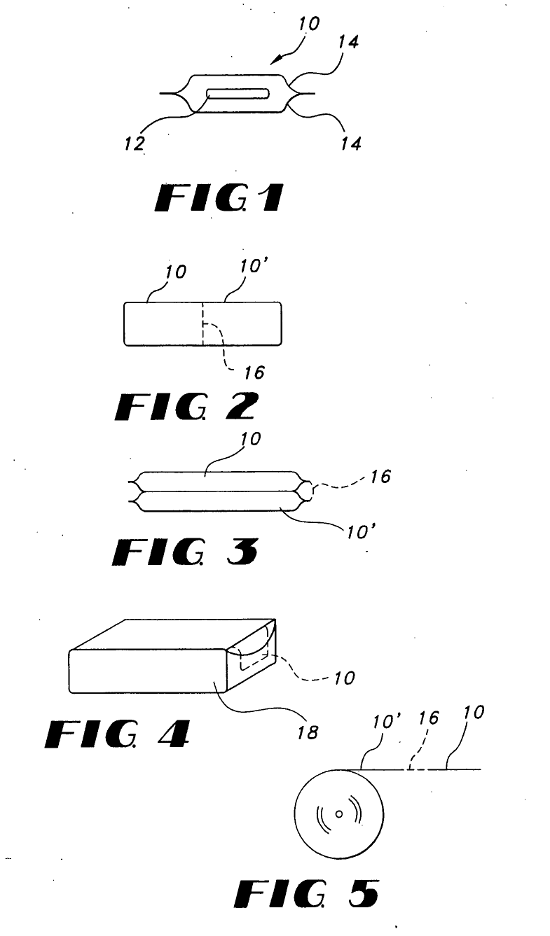

The films of the present invention are desirably packaged in sealed, air and moisture resistant packages to protect the active from exposure oxidation, hydrolysis, volatilization and interaction with the environment. Referring to FIG. 1, a packaged pharmaceutical dosage unit 10, includes each film 12 individually wrapped in a pouch or between foil and/or plastic laminate sheets 14. As depicted in FIG. 2, the pouches 10, 10′ can be linked together with tearable or perforated joints 16. The pouches 10, 10′ may be packaged in a roll as depicted in FIG. 5 or stacked as shown in FIG. 3 and sold in a dispenser 18 as shown in FIG. 4. The dispenser may contain a full supply of the medication typically prescribed for the intended therapy, but due to the thinness of the film and package, is smaller and more convenient than traditional bottles used for tablets, capsules and liquids. Moreover, the films of the present invention dissolve instantly upon contact with saliva or mucosal membrane areas, eliminating the need to wash the dose down with water.

Desirably, a series of such unit doses are packaged together in accordance with the prescribed regimen or treatment, e.g., a 10-90 day supply, depending on the particular therapy. The individual films can be packaged on a backing and peeled off for use.

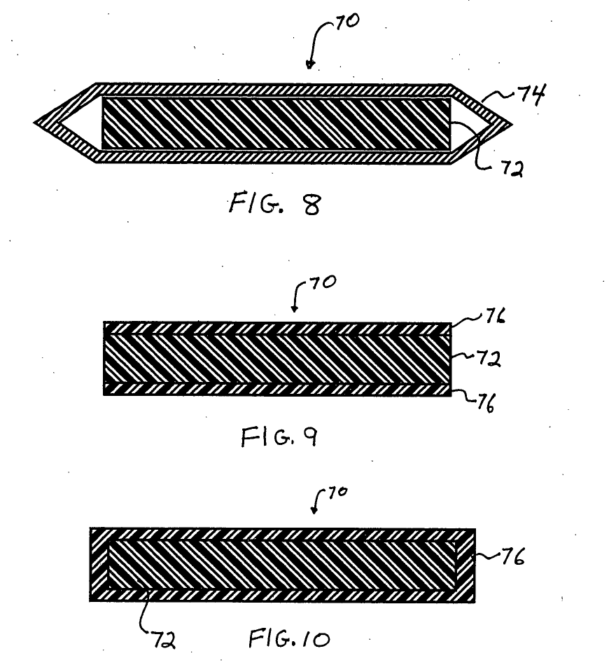

Moreover, the volatile decongestant-containing film of the present invention may be contained or sealed, either totally or partially, in a barrier to minimize decongestant loss. For example, FIG. 8 depicts an article manufacture 70 having a volatile decongestant-containing film 72 sealed with a barrier 74. The barrier 74 may be of thin construction, such as a foil. The foil may be a metal foil, a plastic foil, a paper foil, including a coated or laminated paper foil, and combinations thereof. Additionally, the barrier 74 may be made from film-forming compositions of the present invention. Desirably, such compositions are fast dissolving and essentially free of volatile decongestants.

The film 72 need not be totally contained or encompassed with the barrier 74. For example, barrier 76 may cover the opposed surfaces of film 72, as depicted in FIG. 9. Additionally, the film 72 may be totally contained within barrier 76 without substantially free air space, as depicted in FIG. 10. Barrier 76 may be selected from any of the above-described barrier materials. Desirably, the barrier 76 is a dried film composition of the present invention which is substantially free of volatile decongestants.

Rheology and Films Properties

For the purposes of the present invention the term non-self-aggregating uniform heterogeneity refers to the ability of the films of the present invention, which are formed from one or more components in addition to a polar solvent, to provide a substantially reduced occurrence of, i.e. little or no, aggregation or conglomeration of components within the film as is normally experienced when films are formed by conventional drying methods such as a high-temperature air-bath using a drying oven, drying tunnel, vacuum drier, or other such drying equipment. The term heterogeneity, as used in the present invention, includes films that will incorporate a single component, such as a polymer, as well as combinations of components, such as a polymer and an active. Uniform heterogeneity includes the substantial absence of aggregates or conglomerates as is common in conventional mixing and heat drying methods used to form films.

Furthermore, the films of the present invention have a substantially uniform thickness, which is also not provided by the use of conventional drying methods used for drying water-based polymer systems. The absence of a uniform thickness detrimentally affects uniformity of component distribution throughout the area of a given film.

The film products of the present invention are produced by a combination of a properly selected polymer and a polar solvent, optionally including an active ingredient as well as other fillers known in the art. These films provide a non-self-aggregating uniform heterogeneity of the components within them by utilizing a selected casting or deposition method and a controlled drying process. Examples of controlled drying processes include, but are not limited to, the use of the apparatus disclosed in U.S. Pat. No. 4,631,837 to Magoon (“Magoon”), herein incorporated by reference, as well as hot air impingement across the bottom substrate and bottom heating plates. Another drying technique for obtaining the films of the present invention is controlled radiation drying, in the absence of uncontrolled air currents, such as infrared and radio frequency radiation (i.e. microwaves).

The objective of the drying process is to provide a method of drying the films that avoids complications, such as the noted “rippling” effect, that are associated with conventional drying methods and which initially dry the upper surface of the film, trapping moisture inside. In conventional oven drying methods, as the moisture trapped inside subsequently evaporates, the top surface is altered by being ripped open and then reformed. These complications are avoided by the present invention, and a uniform film is provided by drying the bottom surface of the film first or otherwise preventing the formation of polymer film formation (skin) on the top surface of the film prior to drying the depth of the film. This may be achieved by applying heat to the bottom surface of the film with substantially no top air flow, or alternatively by the introduction of controlled microwaves to evaporate the water or other polar solvent within the film, again with substantially no top air flow. Yet alternatively, drying may be achieved by using balanced fluid flow, such as balanced air flow, where the bottom and top air flows are controlled to provide a uniform film. In such a case, the air flow directed at the top of the film should not create a condition which would cause movement of particles present in the wet film, due to forces generated by the air currents. Additionally, air currents directed at the bottom of the film should desirably be controlled such that the film does not lift up due to forces from the air. Uncontrolled air currents, either above or below the film, can create non-uniformity in the final film products. The humidity level of the area surrounding the top surface may also be appropriately adjusted to prevent premature closure or skinning of the polymer surface.

This manner of drying the films provides several advantages. Among these are the faster drying times and a more uniform surface of the film, as well as uniform distribution of components for any given area in the film. In addition, the faster drying time allows viscosity to quickly build within the film, further encouraging a uniform distribution of components and decrease in aggregation of components in the final film product. Desirably, the drying of the film will occur within about ten minutes or fewer, or more desirably within about five minutes or fewer.

The present invention yields exceptionally uniform film products when attention is paid to reducing the aggregation of the compositional components. By avoiding the introduction of and eliminating excessive air in the mixing process, selecting polymers and solvents to provide a controllable viscosity and by drying the film in a rapid manner from the bottom up, such films result.

The products and processes of the present invention rely on the interaction among various steps of the production of the films in order to provide films that substantially reduce the self-aggregation of the components within the films. Specifically, these steps include the particular method used to form the film, making the composition mixture to prevent air bubble inclusions, controlling the viscosity of the film-forming composition and the method of drying the film. More particularly, a greater viscosity of components in the mixture is particularly useful when the active is not soluble in the selected polar solvent in order to prevent the active from settling out. However, the viscosity must not be too great as to hinder or prevent the chosen method of casting, which desirably includes reverse roll coating due to its ability to provide a film of substantially consistent thickness.

In addition to the viscosity of the film or film-forming components or matrix, there are other considerations taken into account by the present invention for achieving desirable film uniformity. For example, stable suspensions are achieved which prevent solid (such as drug particles) sedimentation in non-colloidal applications. One approach provided by the present invention is to balance the density of the particulate (ρₚ) and the liquid phase (ρₗ) and increase the viscosity of the liquid phase (μ). For an isolated particle, Stokes law relates the terminal settling velocity (Vo) of a rigid spherical body of radius (r) in a viscous fluid, as follows:

Vₒ=(2gr^{r})(ρₚ−ρₗ)/9μ

At high particle concentrations, however, the local particle concentration will affect the local viscosity and density. The viscosity of the suspension is a strong function of solids volume fraction, and particle-particle and particle-liquid interactions will further hinder settling velocity.

Stokian analyses has shown that the incorporation of a third phase, dispersed air or nitrogen, for example, promotes suspension stability. Further, increasing the number of particles leads to a hindered settling effect based on the solids volume fraction. In dilute particle suspensions, the rate of sedimentation, v, can be expressed as:

v/Vₒ=1/(1+κφ)

where κ=a constant, and Ω is the volume fraction of the dispersed phase. More particles suspended in the liquid phase results in decreased velocity. Particle geometry is also an important factor since the particle dimensions will affect particle-particle flow interactions.

Similarly, the viscosity of the suspension is dependent on the volume fraction of dispersed solids. For dilute suspensions of non-interaction spherical particles, an expression for the suspension viscosity can be expressed as:

μ/μ=1+2.5φ

where μhd o is the viscosity of the continuous phase and Ω is the solids volume fraction. At higher volume fractions, the viscosity of the dispersion can be expressed as

μ/μₒ=1+2.5φ+C₁φ²+C₂φ³+. . .

where C is a constant.

The viscosity of the liquid phase is critical and is desirably modified by customizing the liquid composition to a viscoelastic non-Newtonian fluid with low yield stress values. This is the equivalent of producing a high viscosity continuous phase at rest. Formation of a viscoelastic or a highly structured fluid phase provides additional resistive forces to particle sedimentation. Further, flocculation or aggregation can be controlled minimizing particle- particle interactions. The net effect would be the preservation of a homogeneous dispersed phase.

The addition of hydrocolloids to the aqueous phase of the suspension increases viscosity, may produce viscoelasticity and can impart stability depending on the type of hydrocolloid, its concentration and the particle composition, geometry, size, and volume fraction. The particle size distribution of the dispersed phase needs to be controlled by selecting the smallest realistic particle size in the high viscosity medium, i.e., <500 μm. The presence of a slight yield stress or elastic body at low shear rates may also induce permanent stability regardless of the apparent viscosity. The critical particle diameter can be calculated from the yield stress values. In the case of isolated spherical particles, the maximum shear stress developed in settling through a medium of given viscosity can be given as

τₘₐₓ=3Vμ/2r

For pseudoplastic fluids, the viscosity in this shear stress regime may well be the zero shear rate viscosity at the Newtonian plateau.

A stable suspension is an important characteristic for the manufacture of a pre-mix composition which is to be fed into the film casting machinery film, as well as the maintenance of this stability in the wet film stage until sufficient drying has occurred to lock-in the particles and matrix into a sufficiently solid form such that uniformity is maintained. For viscoelastic fluid systems, a rheology that yields stable suspensions for extended time period, such as 24 hours, must be balanced with the requirements of high-speed film casting operations. A desirable property for the films is shear thinning or pseudoplasticity, whereby the viscosity decreases with increasing shear rate. Time dependent shear effects such as thixotropy are also advantageous. Structural recovery and shear thinning behavior are important properties, as is the ability for the film to self-level as it is formed.

The rheology requirements for the inventive compositions and films are quite severe. This is due to the need to produce a stable suspension of particles, for example 30-60 wt %, in a viscoelastic fluid matrix with acceptable viscosity values throughout a broad shear rate range. During mixing, pumping, and film casting, shear rates in the range of 10-10⁵ sec.⁻¹ may be experienced and pseudoplasticity is the preferred embodiment.

In film casting or coating, rheology is also a defining factor with respect to the ability to form films with the desired uniformity. Shear viscosity, extensional viscosity, viscoelasticity, structural recovery will influence the quality of the film. As an illustrative example, the leveling of shear-thinning pseudoplastic fluids has been derived as

α^{(n-1/n)}=αₒ^{(n-1/n)}−((n−1)/(2n−1)) (τ/K)^{1/n}(2π/λ)^{(3+n)/n}h^{(2n+1)/n}t

where α is the surface wave amplitude, αₒ is the initial amplitude, λ is the wavelength of the surface roughness, and both “n” and “K” are viscosity power law indices. In this example, leveling behavior is related to viscosity, increasing as n decreases, and decreasing with increasing K.

Desirably, the films or film-forming compositions of the present invention have a very rapid structural recovery, i.e. as the film is formed during processing, it doesn't fall apart or become discontinuous in its structure and compositional uniformity. Such very rapid structural recovery retards particle settling and sedimentation. Moreover, the films or film-forming compositions of the present invention are desirably shear-thinning pseudoplastic fluids. Such fluids with consideration of properties, such as viscosity and elasticity, promote thin film formation and uniformity.

Thus, uniformity in the mixture of components depends upon numerous variables. As described herein, viscosity of the components, the mixing techniques and the Theological properties of the resultant mixed composition and wet casted film are important aspects of the present invention. Additionally, control of particle size and particle shape are further considerations. Desirably, the size of the particulate a particle size of 150 microns or less, for example 100 microns or less. Moreover, such particles may be spherical, substantially spherical, or non-spherical, such as irregularly shaped particles or ellipsoidally shaped particles. Ellipsoidally shaped particles or ellipsoids are desirable because of their ability to maintain uniformity in the film-forming matrix as they tend to settle to a lesser degree as compared to spherical particles.

Although a variety of different polymers may be used, it is desired to select polymers to provide a desired viscosity of the mixture prior to drying. For example, if the active or other components are not soluble in the selected solvent, a polymer that will provide a greater viscosity is desired to assist in maintaining uniformity. On the other hand, if the components are soluble in the solvent, a polymer that provides a lower viscosity may be preferred.

The polymer plays an important role in affecting the viscosity of the film. Viscosity is one property of a liquid that controls the stability of the active in an emulsion, a colloid or a suspension. Generally the viscosity of the matrix will vary from about 400 cps (“cps” or “centipoise”) to about 100,000 cps, preferably from about 800 cps to about 60,000 cps, and most preferably from about 1,000 cps to about 40,000 cps. Desirably, the viscosity of the film-forming matrix will rapidly increase upon initiation of the drying process.

The viscosity may be adjusted based on the selected active depending on the other components within the matrix. For example, if the component is not soluble within the selected solvent, a proper viscosity may be selected to prevent the component from settling which would adversely affect the uniformity of the resulting film. The viscosity may be adjusted in different ways. To increase viscosity of the film matrix, the polymer may be chosen of a higher molecular weight or crosslinkers may be added, such as salts of calcium, sodium and potassium. The viscosity may also be adjusted by adjusting the temperature or by adding a viscosity increasing component. Components that will increase the viscosity or stabilize the emulsion/suspension include higher molecular weight polymers and polysaccharides and gums, which include without limitation, alginate, carrageenan, hydroxypropyl methyl cellulose, locust bean gum, guar gum, xanthan gum, dextran, gum arabic, gellan gum and combinations thereof.

Film Component Mixing:

A number of techniques may be employed in the mixing stage to prevent bubble inclusions in the final film. To provide a composition mixture with substantially no air bubble formation in the final product, anti-foaming or surface-tension reducing agents are employed. Additionally, the speed of the mixture is desirably controlled to prevent cavitation of the mixture in a manner which pulls air into the mix. Finally, air bubble reduction can further be achieved by allowing the mix to stand for a sufficient time for bubbles to escape prior to drying the film. Desirably, the inventive process first forms a masterbatch of film-forming components without active ingredients such as drug particles or volatile materials such as flavor oils. The actives are added to smaller mixes of the masterbatch just prior to casting. Thus, the masterbatch pre-mix can be allowed to stand for a longer time without concern for instability in drug or other ingredients.

When the matrix is formed including the film-forming polymer and polar solvent in addition to any additives and the active ingredient, this may be done in a number of steps. For example, the ingredients may all be added together or a pre-mix may be prepared. The advantage of a pre-mix is that all ingredients except for the active may be combined in advance, with the active added just prior to formation of the film. This is especially important for actives that may degrade with prolonged exposure to water, air or another polar solvent.

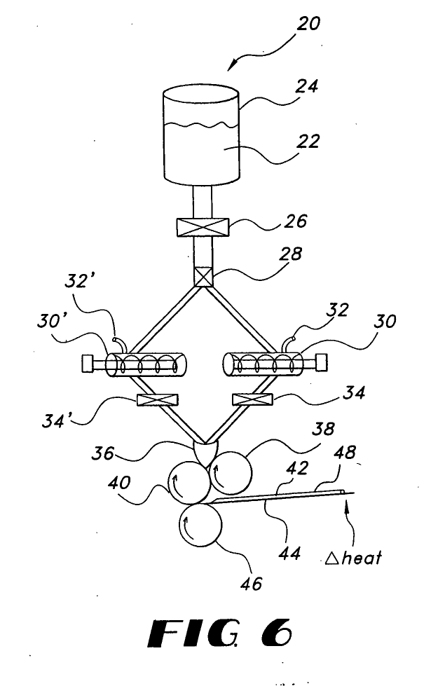

FIG. 6 shows an apparatus 20 suitable for the preparation of a pre-mix, addition of an active and subsequent formation of a film. The pre-mix or master batch 22, which includes the film-forming polymer, polar solvent, and any other additives except a drug active is added to the master batch feed tank 24. The components for pre-mix or master batch 22 are desirably. formed in a mixer (not shown) prior to their addition into the master batch feed tank 24. Then a pre-determined amount of the master batch is controllably fed via a first metering pump 26 and control valve 28 to either or both of the first and second mixers, 30, 30′. The present invention, however, is not limited to the use of two mixers, 30, 30′, and any number of mixers may suitably be used. Moreover, the present invention is not limited to any particular sequencing of the mixers 30, 30′, such as parallel sequencing as depicted in FIG. 6, and other sequencing or arrangements of mixers, such as series or combination of parallel and series, may suitably be used. The required amount of the drug or other ingredient, such as a flavor, is added to the desired mixer through an opening, 32, 32′, in each of the mixers, 30, 30′. Desirably, the residence time of the pre-mix or master batch 22 is minimized in the mixers 30, 30′. While complete dispersion of the drug into the pre-mix or master batch 22 is desirable, excessive residence times may result in leaching or dissolving of the drug, especially in the case for a soluble drug. Thus, the mixers 30, 30′ are often smaller, i.e. lower residence times, as compared to the primary mixers (not shown) used in forming the pre-mix or master batch 22. After the drug has been blended with the master batch pre-mix for a sufficient time to provide a uniform matrix, a specific amount of the uniform matrix is then fed to the pan 36 through the second metering pumps, 34, 34′. The metering roller 38 determines the thickness of the film 42 and applies it to the application roller. The film 42 is finally formed on the substrate 44 and carried away via the support roller 46.

Forming the Film

The films of the present invention must be formed into a sheet prior to drying. After the desired components are combined to form a multi-component matrix, including the polymer, water, and an active or other components as desired, the combination is formed into a sheet or film, by any method known in the art such as extrusion, coating, spreading, casting or drawing the multi-component matrix. If a multi-layered film is desired, this may be accomplished by co-extruding more than one combination of components which may be of the same or different composition. A multi-layered film may also be achieved by coating, spreading, or casting a combination onto an already formed film layer.

Although a variety of different film-forming techniques may be used, it is desirable to select a method that will provide a flexible film, such as reverse roll coating. The flexibility of the film allows for the sheets of film to be rolled and transported for storage or prior to being cut into individual dosage forms. Desirably, the films will also be self-supporting or in other words able to maintain their integrity and structure in the absence of a separate support. Furthermore, the films of the present invention may be selected of materials that are edible or ingestible.

Coating or casting methods are particularly useful for the purpose of forming the films of the present invention. Specific examples include reverse roll coating, gravure coating, immersion or dip coating, metering rod or meyer bar coating, slot die or extrusion coating, gap or knife over roll coating, air knife coating, curtain coating, or combinations thereof, especially when a multi-layered film is desired.

Roll coating, or more specifically reverse roll coating, is particularly desired when forming films in accordance with the present invention. This procedure provides excellent control and uniformity of the resulting films, which is desired in the present invention. In this procedure, the coating material is measured onto the applicator roller by the precision setting of the gap between the upper metering roller and the application roller below it. The coating is transferred from the application roller to the substrate as it passes around the support roller adjacent to the application roller. Both three roll and four roll processes are common.

The gravure coating process relies on an engraved roller running in a coating bath, which fills the engraved dots or lines of the roller with the coating material. The excess coating on the roller is wiped off by a doctor blade and the coating is then deposited onto the substrate as it passes between the engraved roller and a pressure roller.

Offset Gravure is common, where the coating is deposited on an intermediate roller before transfer to the substrate.

In the simple process of immersion or dip coating, the substrate is dipped into a bath of the coating, which is normally of a low viscosity to enable the coating to run back into the bath as the substrate emerges.

In the metering rod coating process, an excess of the coating is deposited onto the substrate as it passes over the bath roller. The wire-wound metering rod, sometimes known as a Meyer Bar, allows the desired quantity of the coating to remain on the substrate. The quantity is determined by the diameter of the wire used on the rod.

In the slot die process, the coating is squeezed out by gravity or under pressure through a slot and onto the substrate. If the coating is 100% solids, the process is termed “Extrusion” and in this case, the line speed is frequently much faster than the speed of the extrusion. This enables coatings to be considerably thinner than the width of the slot.

The gap or knife over roll process relies on a coating being applied to the substrate which then passes through a “gap” between a “knife” and a support roller. As the coating and substrate pass through, the excess is scraped off.

Air knife coating is where the coating is applied to the substrate and the excess is “blown off” by a powerful jet from the air knife. This procedure is useful for aqueous coatings.

In the curtain coating process, a bath with a slot in the base allows a continuous curtain of the coating to fall into the gap between two conveyors. The object to be coated is passed along the conveyor at a controlled speed and so receives the coating on its upper face.

Drying the Film

While the proper viscosity, uniformity in mixture and stable suspension of particles, and casting method are important in the initial steps of forming the film to promote uniformity, the method of drying the wet film is also important. Although these parameters and properties assist uniformity initially, a controlled rapid drying process ensures that the uniformity will be maintained until the film is dry. A controlled drying process is particularly important when, in the absence of a viscosity increasing composition or a composition in which the viscosity is controlled, for example by the selection of the polymer, the components within the film may have an increased tendency to aggregate or conglomerate. An alternative method of forming a film with an accurate dosage, that would not necessitate the controlled drying process, would be to cast the films on a predetermined well. With this method, although the components may aggregate, this will not result in the migration of the active to an adjacent dosage form, since each well may define the dosage unit per se.

When a controlled or rapid drying process is desired, this may be through a variety of methods. A variety of methods may be used including those that require the application of heat. The liquid carriers are removed from the film in a manner such that the uniformity, or more specifically, the non-self-aggregating uniform heterogeneity, that is obtained in the wet film is maintained.

Desirably, the film is dried from the bottom of the film to the top of the film. Substantially no air flow is present across the top of the film during its initial setting period, during which a solid, visco-elastic structure is formed. This can take place within the first few minutes, e.g. about the first ½ minute to about the first 4 minutes of the drying process. Controlling the drying in this manner, prevents the destruction and reformation of the film's top surface, which results from conventional drying methods. This is accomplished by forming the film and placing it on the top side of a surface having top and bottom sides. Then, heat is initially applied to the bottom side of the film to provide the necessary energy to evaporate or otherwise remove the liquid carrier. The films dried in this manner dry more quickly and evenly as compared to air-dried films, or those dried by conventional drying means. In contrast to an air-dried film that dries first at the top and edges, the films dried by applying heat to the bottom dry simultaneously at the center as well as at the edges. This also prevents settling of ingredients that occurs with films dried by conventional means.

The temperature at which the films are dried is about 100° C. or less, desirably about 90° C. or less, and most desirably about 40° C. or less.

Another method of controlling the drying process, which may be used alone or in combination with other controlled methods as disclosed above includes controlling and modifying the humidity within the drying apparatus where the film is being dried. In this manner, the premature drying of the top surface of the film is avoided.

A specific example of an appropriate drying method is that disclosed by Magoon. Magoon is specifically directed toward a method of drying fruit pulp. However, the present inventors have adapted this process toward the preparation of thin films.

The method and apparatus of Magoon are based on an interesting property of water. Although water transmits energy by conduction and convection both within and to its surroundings, water only radiates energy within and to water. Therefore, the apparatus of Magoon includes a surface onto which the fruit pulp is placed that is transparent to infrared radiation. The underside of the surface is in contact with a temperature controlled water bath. The water bath temperature is desirably controlled at a temperature slightly below the boiling temperature of water. When the wet fruit pulp is placed on the surface of the apparatus, this creates a “refractance window.” This means that infrared energy is permitted to radiate through the surface only to the area on the surface occupied by the fruit pulp, and only until the fruit pulp is dry. The apparatus of Magoon provides the films of the present invention with an efficient drying time reducing the instance of aggregation of the components of the film.

The films may initially have a thickness of about 500 μm to about 1,500 μm, or about 20 mils to about 60 mils, and when dried have a thickness from about 3 μm to about 250 μm, or about 0.1 mils to about 10 mils. Desirably, the dried films will have a thickness of about 2 mils to about 8 mils, and more desirably, from about 3 mils to about 6 mils.

The wet film is then dried using controlled bottom drying or controlled microwave drying, desirably in the absence of external air currents or heat on the top (exposed) surface of the film 48 as described herein. Controlled bottom drying or controlled microwave drying advantageously allows for vapor release from the film without the disadvantages of the prior art. Conventional convection air drying from the top is not employed because it initiates drying at the top uppermost portion of the film, thereby forming a barrier against fluid flow, such as the evaporative vapors, and thermal flow, such as the thermal energy for drying. Such dried upper portions serve as a barrier to further vapor release as the portions beneath are dried, which results in non-uniform films. As previously mentioned some top air flow can be used to aid the drying of the films of the present invention, but it must not create a condition that would cause particle movement or a rippling effect in the film, both of which would result in non-uniformity. If top air is employed, it is balanced with the bottom air drying to avoid non-uniformity and prevent film lift-up on the carrier belt. A balance top and bottom air flow may be suitable where the bottom air flow functions as the major source of drying and the top air flow is the minor source of drying. The advantage of some top air flow is to move the exiting vapors away from the film thereby aiding in the overall drying process. The use of any top air flow or top drying, however, must be balanced by a number of factors including, but not limited, to rheological properties of the composition and mechanical aspects of the processing. Any top fluid flow, such as air, also must not overcome the inherent viscosity of the film-forming composition. In other words, the top air flow cannot break, distort or otherwise physically disturb the surface of the composition. Moreover, air velocities are desirably below the yield values of the film, i.e., below any force level that can move the liquids in the film-forming compositions. For thin or low viscosity compositions, low air velocity must be used. For thick or high viscosity compositions, higher air velocities may be used. Furthermore, air velocities are desirable low so as to avoid any lifting or other movement of the film formed from the compositions.

Moreover, the films of the present invention may contain particles that are sensitive to temperature, such as flavors, which may be volatile, or drugs, which may have a low degradation temperature. In such cases, the drying temperature may be decreased while increasing the drying time to adequately dry the uniform films of the present invention. Furthermore, bottom drying also tends to result in a lower internal film temperature as compared to top drying. In bottom drying, the evaporating vapors more readily carry heat away from the film as compared to top drying which lowers the internal film temperature. Such lower internal film temperatures often result in decreased drug degradation and decreased loss of certain volatiles, such as flavors.

Furthermore, particles or particulates may be added to the film-forming composition or matrix after the composition or matrix is cast into a film. For example, particles may be added to the film 42 prior to the drying of the film 42. Particles may be controllably metered to the film and disposed onto the film through a suitable technique, such as through the use of a doctor blade (not shown) which is a device which marginally or softly touches the surface of the film and controllably disposes the particles onto the film surface. Other suitable, but non-limiting, techniques include the use of an additional roller to place the particles on the film surface, spraying the particles onto the film surface, and the like. The particles may be placed on either or both of the opposed film surfaces, i.e., the top and/or bottom film surfaces. Desirably, the particles are securably disposed onto the film, such as being embedded into the film. Moreover, such particles are desirably not fully encased or fully embedded into the film, but remain exposed to the surface of the film, such as in the case where the particles are partially embedded or partially encased.

The particles may be any useful organoleptic agent, cosmetic agent, pharmaceutical agent, or combinations thereof. Desirably, the pharmaceutical agent is a taste-masked or a controlled-release pharmaceutical agent. Useful organoleptic agents include flavors and sweeteners. Useful cosmetic agents include breath freshening or decongestant agents, such as menthol, including menthol crystals.

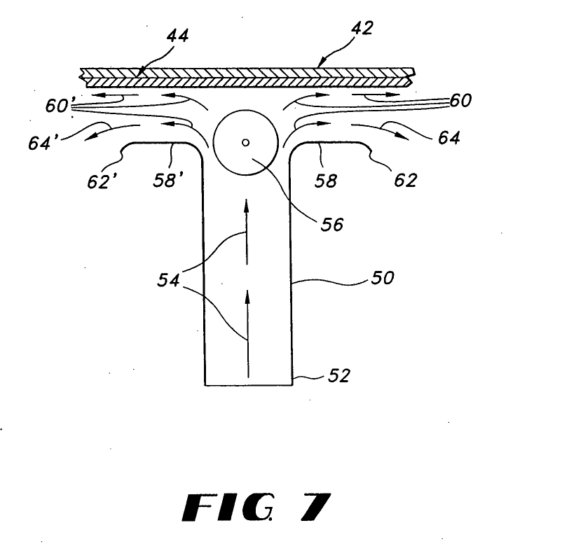

Although the inventive process is not limited to any particular apparatus for the above-described desirable drying, one particular useful drying apparatus 50 is depicted in FIG. 7. Drying apparatus 50 is a nozzle arrangement for directing hot fluid, such as but not limited to hot air, towards the bottom of the film 42 which is disposed on substrate 44. Hot air enters the entrance end 52 of the drying apparatus and travels vertically upward, as depicted by vectors 54, towards air deflector 56. The air deflector 56 redirects the air movement to minimize upward force on the film 42. As depicted in FIG. 7, the air is tangentially directed, as indicated by vectors 60 and 60′, as the air passes by air deflector 56 and enters and travels through chamber portions 58 and 58′ of the drying apparatus 50. With the hot air flow being substantially tangential to the film 42, lifting of the film as it is being dried is thereby minimized. While the air deflector 56 is depicted as a roller, other devices and geometries for deflecting air or hot fluid may suitable be used. Furthermore, the exit ends 62 and 62′ of the drying apparatus 50 are flared downwardly. Such downward flaring provides a downward force or downward velocity vector, as indicated by vectors 64 and 64′, which tend to provide a pulling or drag effect of the film 42 to prevent lifting of the film 42. Lifting of the film 42 may not only result in non-uniformity in the film or otherwise, but may also result in non-controlled processing of the film 42 as the film 42 and/or substrate 44 lift away from the processing equipment.

Monitoring and control of the thickness of the film also contributes to the production of a uniform film by providing a film of uniform thickness. The thickness of the film may be monitored with gauges such as Beta Gauges. A gauge may be coupled to another gauge at the end of the drying apparatus, i.e. drying oven or tunnel, to communicate through feedback loops to control and adjust the opening in the coating apparatus, resulting in control of uniform film thickness.

The film products are generally formed by combining a properly selected polymer and polar solvent, as well as any active ingredient or filler as desired. Desirably, the solvent content of the combination is at least about 30% by weight of the total combination. The matrix formed by this combination is formed into a film, desirably by roll coating, and then dried, desirably by a rapid and controlled drying process to maintain the uniformity of the film, more specifically, a non-self-aggregating uniform heterogeneity. The resulting film will desirably contain less than about 10% by weight solvent, more desirably less than about 8% by weight solvent, even more desirably less than about 6% by weight solvent and most desirably less than about 2%. The solvent may be water, a polar organic solvent including, but not limited to, ethanol, isopropanol, acetone, methylene chloride, or any combination thereof.

It has also been unexpectedly discovered that high temperature fat materials, e.g. M.P. 55° C. or greater, can be used to encapsulate dry particles before or after enteric coating. The drying process temperatures are sufficiently rapid and low, and evaporative cooling effect as a result of water vapor loss is sufficiently high enough, that the fat does not appreciably melt.

Consideration of the above discussed parameters, such as but not limited to rheology properties, viscosity, mixing method, casting method and drying method, also impact material selection for the different components of the present invention. Furthermore, such consideration with proper material selection provides the compositions of the present invention, including a pharmaceutical and/or cosmetic dosage form or film product having no more than a 10% variance of a pharmaceutical and/or cosmetic active per unit area. In other words, the uniformity of the present invention is determined by the presence of no more than a 10% by weight of pharmaceutical and/or cosmetic variance throughout the matrix. Desirably, the variance is less than 5% by weight, less than 2% by weight, less than 1% by weight, or less than 0.5% by weight.

The following non-limiting examples are intended to further illustrate the present invention.

### EXAMPLES

### Preparation of Menthol-Containing Film by Depositing Menthol Crystals onto the Film or onto a Film-forming Composition

A film-forming composition, Composition A in Table 1 below, was prepared and mixed under vacuum to remove air bubbles. In further detail, plasticizer (propylene glycol), glycerin, and anti-foam agent (polydimethylsiloxane emulsion) were added to water with stirring over a short period of time of about 15 minutes. Hydroxypropylmethyl cellulose (Methocel™ E15), hydropropyl cellulose, starch, precipitated calcium carbonate, and sweetner/tastemasking flavor (Sucralose and Magna Sweet) were added to the above mixture with mixing or stirring. The stirring was set at 100 rpm using an axial impeller. Stirring continued for another 36 minutes with a vacuum being applied towards the end to remove air bubbles.

TABLE 1

| Film-forming Polymer Composition | Composition A |
|---|---|
| Ingredient | (weight parts) |
| Hydroxypropylmethyl cellulose | 6.75 |
| Hydroxypropyl cellulose | 6.75 |
| Starch | 2.5 |
| Sweetener/Tastemasking Flavor | 1.06 |
| Precipitated Calcium Carbonate | 12.44 |
| Glycerin | 1. |
| Plasticizer | 2. |
| Antifoam agent | 0.75 |
| Water | 65. |

Part of the solution was cast into a film and dried at 90° C. for about 9 minutes. The dried film has about 3.0 percent moisture. The film quickly dissolved in the mouth.

The solution was also cast into additional films with menthol crystals being added to the surface of the cast film-forming composition. The composition was dried into a film. The addition of the menthol caused surface imperfections in the film. During drying the menthol crystals melted and/or sublimed. Menthol crystals were also added after drying the composition and after particle drying of the composition, but menthol melting and/or sublimation caused surface imperfections and loss of menthol product.

### Preparation of Menthol-Containing Film by Depositing Menthol Crystals into the Film-forming Composition

A film-forming composition, Composition B in Table 2 below, was prepared and mixed under vacuum to remove air bubbles. In further detail, anti-foam agent (polydimethylsiloxane emulsion) and to water were combined with stirring over a short period of time. Hydroxypropylmethyl cellulose (Methocel™ E15), hydropropyl cellulose, starch, precipitated calcium carbonate, and sweetner/tastemasking flavor (Sucralose and Magna Sweet) were added to the above mixture with mixing or stirring. The stirring was set at 100 rpm using an axial impeller. Stirring continued for about another 40 minutes with a vacuum being applied towards the end to remove air bubbles. Menthol crystals, plasticizer (propylene glycol), and glycerin were added under partial vacuum with stirring at 100 rpm. Stirring continued for a very short time of about 2 minutes. The film-forming composition had about 35 weight percent solids:

TABLE 2

| Film-forming Polymer Composition | Composition B |
|---|---|
| Ingredient | (weight parts) |
| Hydroxypropylmethyl cellulose | 6.75 |
| Hydroxypropyl cellulose | 6.75 |
| Starch | 2.5 |
| Sweetener/Tastemasking Flavor | 1.06 |
| Precipitated Calcium Carbonate | 10.69 |
| Glycerin | 1. |
| Plasticizer | 2. |
| Antifoam agent | 0.75 |
| Menthol crystals | 3.5 |
| Water | 65. |

Part of the solution was cast into a film and dried at 90° C. for about 9 minutes. The dried film has about 3.0 percent moisture. The film quickly dissolved in the mouth and had god decongestant action.

Additional water was added to part of the solution to yield a solution with 30 weight percent solids. This solution was stirred at 100 rpm for about 8 minutes with a partial vacuum being applied towards the end. The solution was also cast into a film and dried at 90° C. for about 9 minutes. The dried film has about 3.6 percent moisture. The film quickly dissolved in the mouth and had good decongestant action. The film composition with 30% solids cast into the film in an easier manner than the

### Preparation of Menthol-Containing Film Free of Plasticizers by Depositing Menthol Crystals into the Film-forming Composition

A film-forming composition, Composition C in Table 3 below, was prepared and mixed under vacuum to remove air bubbles. In further detail, anti-foam agent (polydimethylsiloxane emulsion) and to water were combined with stirring over a short period of time. Hydroxypropylmethyl cellulose (Methocel™ E15), hydropropyl cellulose, starch, precipitated calcium carbonate, and sweetner/tastemasking flavor (Sucralose and Magna Sweet) were added to the above mixture with mixing or stirring. The stirring was set at 100 rpm using an axial impeller. Stirring continued for about another 40 minutes with a vacuum being applied towards the end to remove air bubbles. Menthol crystals were added under partial vacuum with stirring at 100 rpm. A minor amount of water also added. Stirring continued for a very short time of about 2 minutes.

TABLE 3

| Film-forming Polymer Composition | Composition C |
|---|---|
| Ingredient | (weight parts) |
| Hydroxypropylmethyl cellulose | 5.79 |
| Hydroxypropyl cellulose | 5.79 |
| Starch | 2.14 |
| Sweetener/Tastemasking Flavor | 0.9 |
| Precipitated Calcium Carbonate | 11.73 |
| Antifoam agent | 0.64 |
| Menthol crystals | 3. |
| Water | 70. |

The solution was cast into a film and dried at 90° C. for about 9 minutes. The dried film has about 3.9 percent moisture. The film quickly dissolved in the mouth and had good decongestant action.

Additional water was added to part of the solution to yield a solution with 30 weight percent solids. This solution was stirred at 100 rpm for about 8 minutes with a partial vacuum being applied towards the end. The solution was also cast into a film and dried at 90° C. for about 9 minutes. The dried film has about 3.6 percent moisture. The film, which was free of plasticizers and/or polyalcohols, quickly dissolved in the mouth and had good decongestant action.

### Preparation of Menthol-Containing Film with Additional Volatile Oils

Film-forming compositions, Compositions D and E in Table 4 below, were prepared as described above. The dried film compositions include a volatile decongestant oil, i.e., eucalyptus oil, as follows:

TABLE 4

| Film-forming Polymer Composition | D | E |
|---|---|---|
| Ingredient | (wt. parts) | (wt. parts) |
| Hydroxypropylmethyl cellulose | 34.88 | 32.61 |
| PVP | 11.63 | 10.87 |
| Calcium Carbonate | 11.63 | 10.87 |
| Tween 80 | 12.78 | 13.03 |
| Menthol crystals | 6.98 | 11.52 |
| Eucalyptus oil | 5.23 | 4.78 |
| Sweetener/Tastemasking Flavor | 10.23 | 9.57 |
| Water | 6.63 | 6.74 |

The films quickly dissolved in the mouth and had good decongestant action.

While there have been described what are presently believed to be the certain desirable embodiments of the invention, those skilled in the art will realize that changes and modifications may be made thereto without departing from the spirit of the invention, and it is intended to include all such changes and modifications as fall within the true scope of the invention.

What is claimed is:

1. A volatile decongestant delivery vehicle composition comprising:
(i) a flowable water-soluble film-forming matrix; and
(ii) a particulate volatile decongestant agent uniformly stationed therein.

2. The delivery composition of claim 1, wherein said matrix is a cellulosic material, a gum, a protein, a starch, a glucan, and combinations thereof.

3. The delivery vehicle composition of claim 1, wherein said matrix is selected from the group consisting of carboxymethyl cellulose, methyl cellulose, ethyl cellulose, hydroxyl methyl cellulose, hydroxyethyl cellulose, hydroxypropyl cellulose, hydroxypropylmethyl cellulose, hydroxymethylpropyl cellulose, and combinations thereof.

4. The delivery vehicle composition of claim 1, wherein said matrix is selected from the group consisting of gum arabic, xanthan gum, tragacanth, acacia, carageenan, guar gum, locust bean gum, pectin, alginates and combinations thereof.

5. The delivery vehicle composition of claim 1, wherein said matrix is a starch selected from the group consisting of tapioca, rice, corn, potato, wheat and combinations thereof.

6. The delivery vehicle composition of claim 5, wherein said starch is gelatinized, modified or unmodified.

7. The delivery vehicle composition of claim 1, wherein said matrix is selected from the group consisting of polyvinyl alcohol, polyacrylic acid, polyvinyl pyrrolidone, poly(meth)acrylate, poly(meth)copolymers and combinations thereof.

8. The delivery vehicle composition of claim 1, wherein said matrix is a protein selected from the group consisting of gelatin, zein, gluten, soy protein, soy protein isolate, whey protein, whey protein isolate, casein, levin, collagen and combinations thereof.

9. The delivery vehicle composition of claim 1, wherein said matrix is selected from the group consisting of dextrin, dextran and combinations thereof.

10. The delivery vehicle composition of claim 1, wherein said matrix is selected from the group consisting of chitin, chitosin and combinations thereof.

11. The delivery vehicle composition of claim 1, wherein said matrix is polydextrose, fructose oligomers, and combinations thereof.

12. The delivery vehicle composition of claim 1, wherein said volatile decongestant agent is menthol.

13. The delivery vehicle composition of claim 1, wherein said volatile decongestant agent is menthol crystals.

14. The delivery vehicle composition of claim 1, wherein said volatile decongestant agent is present in amounts of up to about 0.1% to about 60% by weight of the total composition.

15. The delivery vehicle composition of claim 1, further including a decongesting volatile oil.

16. The delivery vehicle composition of claim 24, wherein said volatile oil is an oil selected from the group consisting of eucalyptus oil, menthol oil, pine oil, terpine hydrate oil, and combinations thereof.

17. The delivery vehicle composition of claim 1, wherein said composition is orally or intranasally deliverable.

18. The delivery composition of claim 1, wherein the composition is essentially free of a surfactant.

19. The delivery composition of claims 1 or 18, wherein the composition is essentially free of a plasticizer.

20. The delivery composition of claims 1, 18 or 19, wherein the composition is essentially free of a polyalcohol.

21. A method of preparing a thin film volatile decongestant delivery vehicle comprising:
(a) providing a volatile decongestant agent complex;
(b) combining said complex with a water-soluble polymer and a solvent to form a decongestant mixture with uniform distribution of said complex therein;
(c) casting said mixture onto a planar carrier surface to form a thin film on said carrier surface; and
(d) controllably drying said thin film to form a distribution variance of said complex having less than about 10% variance throughout any given area of said thin film.

22. The method of claim 21, wherein said drying includes applying heat to the bottom of said carrier surface.

23. The method of claim 21, wherein said drying includes applying microwave energy to said film.

24. The method of claim 21, further including the steps of mixing said water-soluble polymer and said solvent to form a pre-decongestant mixture and mixing said pre-decongestant mixture to obtain uniform distribution.

25. The method of claim 24, the said complex is added after mixing said pre-decongestant mixture.

26. The method of claim 25, wherein the time of mixing said pre-decongestant mixture is greater than the time of mixing said decongestant mixture therein.

27. The method of claim 21, wherein the step of providing a volatile decongestant agent complex includes providing menthol crystals.

28. The method of claim 27, wherein the step of providing a volatile decongestant agent complex further includes a decongesting volatile oil.

29. A method of providing decongesting relief comprising:
orally or intranasally delivering the delivery vehicle composition of claim 1.

30. A decongestant article comprising:
the delivery vehicle composition of claim 1; and
an enclosure for said composition.

31. The article of claim 30, wherein said enclosure comprises a foil encompassing said composition.

32. The article of claim 31, where said foil is a metal foil.

33. The article of claim 30, wherein said enclosure comprises an outer film obtained from a flowable water-soluble film-forming matrix, wherein said outer film is essentially free of volatile decongestants.
